(19) United States
(12) Patent Application Publication
(10) Pub. No.: US 2015/0277733 A1
(43) Pub. Date: Oct. 1, 2015

(54) DEVICE FOR ARTIFICIAL RESPIRATION

(71) Applicant: WEINMANN GERAETE FUER MEDIZIN GMBH & CO. KG, Hamburg (DE)

(72) Inventor: Matthias Schwaibold, Karlsruhe (DE)

(21) Appl. No.: 14/670,737

(22) Filed: Mar. 27, 2015

(30) Foreign Application Priority Data
Mar. 28, 2014 (DE) 102014004848.9

(51) Int. Cl. G06F 3/0484; A61M 16/16; A61M 16/06; G06F 3/0482; A61M 16/00

(52) U.S. Cl. CPC G06F 3/04847; G06F 3/0482; G06F 3/04842; A61M 16/0066; A61M 16/06; A61M 16/16; A61M 2205/505; A61M 2205/52; A61M 2205/15; A61M 2205/33

(57) ABSTRACT

The disclosed device serves for artificial respiration and has a blower connected to a control. Both the control and the blower are arranged in a housing. The control is connected to at least one indicating device and also to at least one operating element.

### CROSS-REFERENCE TO RELATED APPLICATIONS

The present application claims priority under 35 U.S.C. §119 of German Patent Application No. 10 2014 004 848.9, filed Mar. 28, 2014, the entire disclosure of which is expressly incorporated by reference herein.

### BACKGROUND OF THE INVENTION

1. Field of the Invention

The invention relates to a device for artificial respiration, which has a blower connected to a control, wherein both the control and the blower are arranged in a housing, and wherein the control is connected to at least one indicating device and also at least one operating element.

2. Discussion of Background Information

Such devices for artificial respiration are typically connected to a supply system for being supplied with power and connected to a respiration mask by means of a respiratory tube. A patient puts on the respiration mask in the area of the face while the artificial respiration is being carried out.

For example, it is known to use such devices for artificial respiration for carrying out CPAP respiration or APAP respiration. In principle, any desired respiration sequences can be preset by the control of the device.

It would be advantageous to have available a device of the type mentioned at the beginning in such a way that improved quality of use is provided.

### SUMMARY OF THE INVENTION

The device for artificial respiration according to the invention comprises a blower connected to a control, both the control and the blower being arranged in a housing, and the control being connected to at least one indicating device and also at least one operating element. The indicating device is formed as a display that has a touchscreen function and, using a symbol-based operating structure, can be used as an input device in such a way that respective symbols visualize an assigned function and that a hierarchically structured operating capability is generated by the control.

In one aspect of the device according to the invention, different languages may be selected by means of the display.

In another aspect of the device according to the invention, a possibility for the selection of different languages may be provided for a patient menu and an expert menu, wherein the language for the menu concerned is selected by means of the input button and the input button is connected by logic circuitry to a language selection menu, in which the user can select the language, wherein the input button is also connected by logic circuitry to the language memory and wherein, after confirmation of the language selected, this language is loaded from the memory and used.

In yet another aspect of the device according to the invention, a number of input buttons configured for receiving a user input may be provided in the region of the display, wherein the display is connected by logic circuitry to the buttons and has a number of assigned information fields, wherein each information field is assigned to a corresponding input button and configured for indicating graphic items of information that specify various functions of the corresponding input buttons, wherein the information field is designed to indicate graphically whether or not a selected value concerning a parameter or a setting of the respirator can be changed by means of at least one input button.

In a still further aspect of the device according to the invention, the display may be connected to an evaluation unit for providing at least one item of qualitative therapy information.

In another aspect of the device according to the invention, the respirator may have an internal data memory for permanently storing operating data.

In another aspect of the device according to the invention, selectable operating data may be storable on a memory card.

In another aspect of the device according to the invention, a number of input buttons configured for receiving a user input may be provided in the region of the display, wherein the display is connected by logic circuitry to the buttons and has a number of assigned information fields, wherein each information field is assigned to a corresponding input button and configured for indicating graphic items of information that specify various functions of the corresponding input buttons, wherein the information field is designed to indicate graphically how many days with memory data are to be transferred to the external memory (an SD card or USB memory).

In another aspect of the device according to the invention, the respirator may be designed for carrying out a leak-tightness test of a respiration mask and a corresponding test result may be visualized in the region of the display.

In another aspect of the device according to the invention, a pressure used while carrying out the test for the leak-tightness of the mask may be preset by means of the display.

In another aspect of the device according to the invention, after actuating the input button, in particular the carrying out of a test procedure for the leak-tightness of the mask may take place during the therapy in the expert menu, wherein a number of input buttons configured for receiving a user input are provided in the region of the display and wherein the display is connected by logic circuitry to the buttons and has a number of assigned information fields, wherein each information field is assigned to a corresponding input button and configured for indicating graphic items of information that specify various functions of the corresponding input buttons, wherein at least one information field is provided with an assigned input button, wherein the information field is designed to indicate graphically whether or not a selected value concerning a parameter or a setting of the respirator can be changed by means of at least one input button and a change of this state can be achieved by means of actuating the input button that is assigned to the information field, wherein, after corresponding actuation of the input button, the information field visualizes a closed state and, after that, at least selected values that concern the operation of the respirator can no longer be changed.

In another aspect of the device according to the invention, at least one energy-saving option may be selectable and may be available for being configured by means of the display.

In another aspect of the device according to the invention, at least one item of information with respect to the function of the device and/or operating the device may be visualized by means of the display.

The method according to the invention for indicating user interface information for a respirator has a blower connected to a control, wherein both the control and the blower are arranged in a housing, and wherein the control is connected to at least one indicating device and also at least one operating element, wherein the indicating device is formed as a display that has a touchscreen function and, using a symbol-based operating structure, can be used as an input device in such a way that respective symbols visualize an assigned function and a hierarchically structured operating capability is generated by the control.

In one aspect of the method according to the invention, a number of input buttons configured for receiving a user input may be provided in the region of the display and the display may be connected by logic circuitry to the buttons and may have a number of assigned information fields, wherein each information field is assigned to a corresponding input button and configured for indicating graphic items of information that specify various functions of the corresponding input buttons, wherein at least one information field with an assigned input button is provided, wherein the information field is designed to indicate graphically whether a selected value concerning a parameter or a setting of the respirator can be changed by means of at least one input button.

### BRIEF DESCRIPTION OF THE DRAWINGS

Exemplary embodiments of the invention are schematically represented in the drawings, in which:

FIG. 1 shows a perspective representation of a device with an obliquely arranged display,

FIG. 2 shows a perspective representation of a modified device configuration,

FIG. 3 shows a perspective representation of a front unit and an assembled blower box,

FIG. 3a shows a further perspective representation of a front unit and an assembled blower box,

FIG. 4 shows a perspective representation of a blower arranged within the blower box, the blower being secured by a peripheral membrane structure,

FIG. 4a shows a further perspective representation of the blower secured by a peripheral membrane,

FIG. 5 shows a perspective representation of a partly disassembled blower box with depicted air conduction,

FIG. 6 shows a representation corresponding to FIG. 5 without depicted air conduction,

FIG. 7 shows a perspective inside view of a housing part,

FIG. 8 shows a perspective representation to illustrate securement of an indicating device formed as a display within the housing by using a frame,

FIG. 9 shows a further representation of the display and the holding frame,

FIG. 10 shows a combined representation of the display and the holding frame,

FIG. 11 shows a further perspective representation of the respirator with an assigned external operating unit and

FIG. 12 shows a plan view of a control board with a pressure measuring tube arranged within the device housing,

FIG. 13 shows a plan view of the display in an inactive state of the respirator, in which automatic testing of the fitted breathing mask for leak-tightness takes place,

FIG. 14 shows a representation of the display in an active basic state, during which the leak-tightness of a mask is tested,

FIG. 15 shows an indication of the display on a menu level for the selection of languages to be used,

FIG. 16 shows a representation of the display on a menu level for the storing of data onto a memory card,

FIG. 17 shows an indication of the display on a menu level for the optional selection of an energy-saving mode,

FIG. 18 shows an indication of the display for the visualization of a qualitative assessment of the therapy carried out,

FIG. 19 shows a number of indications of the display for more detailed assessments of the therapy carried out and

FIG. 20 shows an indication of the display for allowing a selection of demonstration representations that are provided by way of example to be shown for the visualization of different functional modes.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

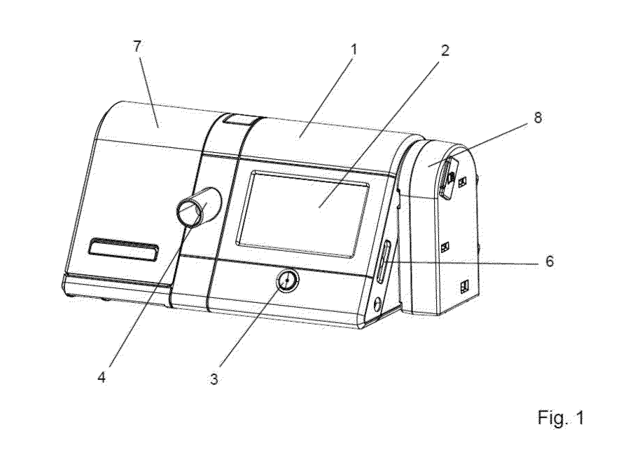

FIG. 1 shows a respirator (1). The respirator (1) is provided with an indicating device (2), at least one operating element (3) and also a tube connection (4).

The tube connection (4) typically serves for connection to a respiratory gas tube (not represented), which is connected in the region of its extent that is away from the tube connection (4) to a breathing mask that is likewise not represented. The breathing mask that is not represented serves for supplying respiratory gas to a patient, who is likewise not represented.

The respirator (1) is typically provided with at least one communication interface (6).

In the case of the exemplary embodiment represented, the respirator (1) is coupled with a respiratory gas humidifier (7). The respirator (1) may be operated optionally both with and without the respiratory gas humidifier (7).

According to the embodiment represented in FIG. 1, the housing of the respirator (1) has a triangular cross-sectional area in a vertical sectional plane.

According to the embodiment in FIG. 1, additional elements (8), which provide at least one further device function, can be coupled to the respirator (1) as and when required.

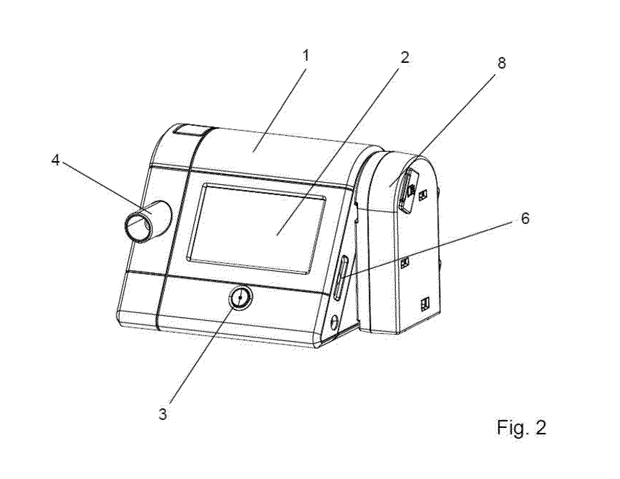

According to the embodiment represented in FIG. 2, in the case of the arrangement of the device according to FIG. 1 the respiratory gas humidifier (7) has been removed from the respirator (1).

It is evident from looking at FIG. 1 together with FIG. 2 that, irrespective of the use of a respiratory gas humidifier (7), the same tube connection (4) can always be used. It is consequently not necessary to change over the respiratory tube according to the device function.

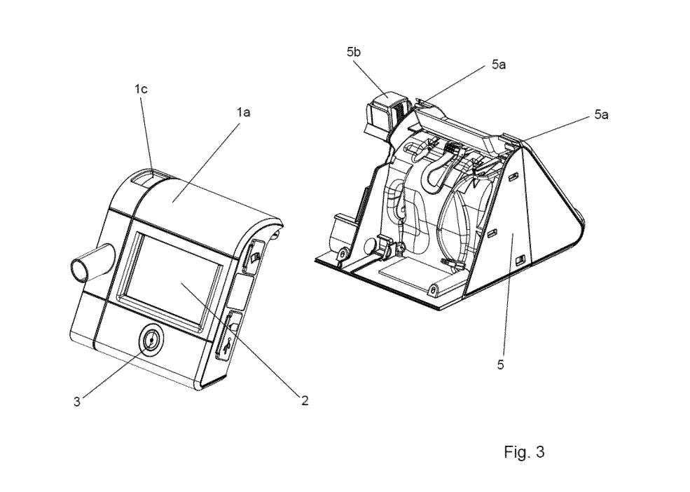

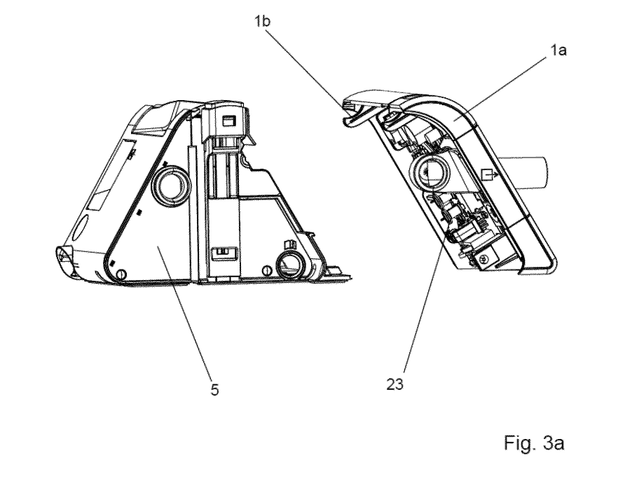

FIG. 3 illustrates that, with regard to its basic mechanical structure, the respirator (1) consists of two components. They are a front unit (1a), with the operating element (3) and the indicating device (2), and the blower box (5), with a front part (16), a supporting part (11) with the blower (9) and a rear part (17). The front part (16) and the rear part (17) enclose an interior device space (18) in the manner of shells. Inserted into the housing parts (16) and (17) are sound-damping materials (16′ and 17′), preferably of a foam material.

FIGS. 3 and 3a show the respirator (1) broken down into the components that are the blower box (5) and the front unit (1a). The easy handling during the assembly of the components is evident from the figures. Apart from the housing, the front unit (1a) includes inter alia an indicating device (2) (for example as a touchscreen (13)), an operating element (3) and a control board (23). The assembly of the front unit (1a) with the blower box (5) is performed by means of hooks (1b) on the front unit (1a), which engage behind regions (5a) of the blower box (5). The front unit is swung in by means of the pivot point comprising the hooks (1b) and the regions (5a). In addition, a catch (5b) arranged on the blower box (5) engages in an opening (1c) in the front unit (1a). To secure it, the front unit (1a) is connected to the blower box (5) by just 2 screws, and thereby forms the housing of the respirator (1). To release the front unit (1a) from the blower box (5), all that is necessary is to loosen the screws and disengage the catch (5b).

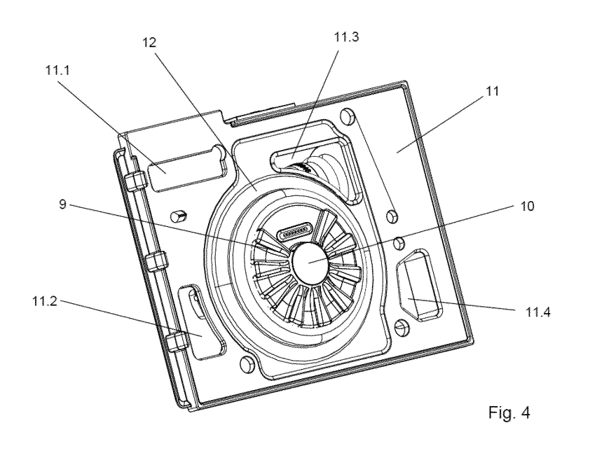

According to the embodiment in FIG. 4, a blower (9) is arranged within the blower box (5). The blower (9) is typically driven by an electric motor (10).

To assist easy assembly and disassembly, the blower (9) may be secured by a supporting part (11), which can be inserted into the blower box (5).

To assist structure-borne sound decoupling, the blower (9) is connected to the supporting part (11) by an elastic coupling element (12). The supporting part (11) and the coupling element (12) are preferably formed as one part. They may for example be made of an elastomeric material. According to a preferred embodiment, the coupling element (12) surrounds the blower (9) in the manner of a frame. Depending on the respective application requirements, the coupling element (12) may be designed in the manner of a ring. The coupling element (12) preferably has a membrane-like design, for example similar to elastic suspension elements that are used in the case of loudspeakers.

The supporting part (11) with the coupling element (12) preferably consists of an elastic material, for example of silicone.

Apart from the coupling element (12), the supporting part (11) includes various air openings, in order to achieve a longer air path, and consequently a further reduction of the noise.

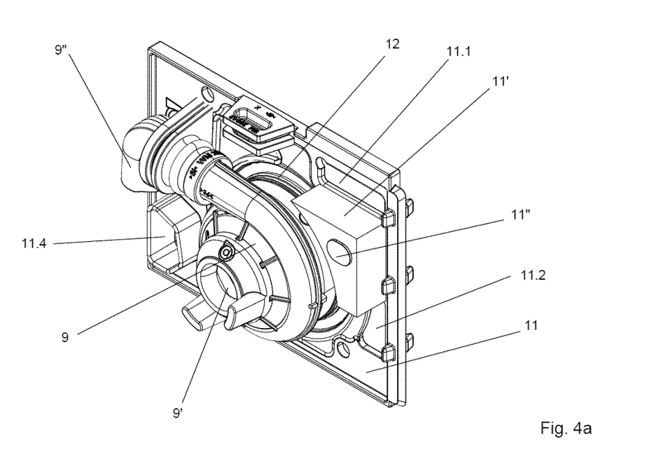

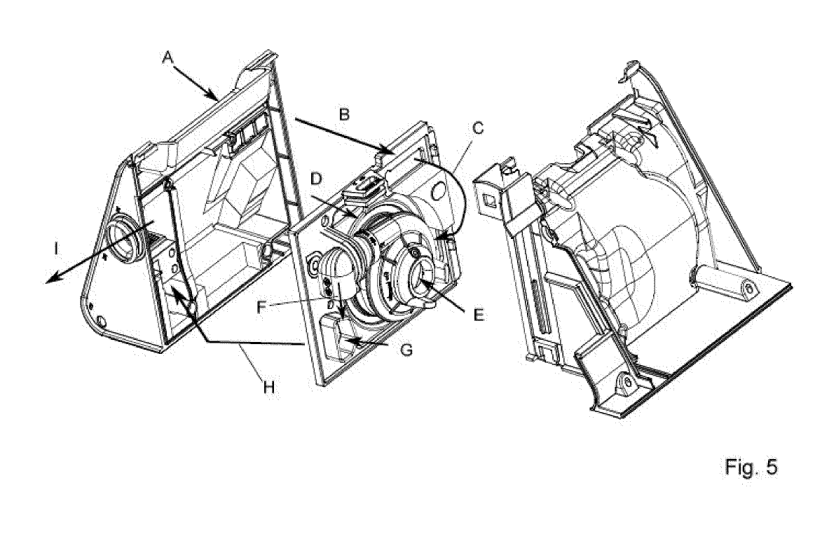

Apart from receiving the blower (9) and the specifically intended gas flow through the interior device space (18) of the blower box (5), which is illustrated in FIG. 5, the supporting part (11) also serves for providing the seal between the housing parts (16) and (17). The outer contour of the supporting part (11) receives the outer contour of the housing parts (16 and 17) and thus provides a seal between the housing parts (16 and 17) in the peripheral region.

FIG. 4a shows the supporting part (11) in a further view. Here, the intake region (9′) and the air outlet (9″) of the blower (9) can be seen. An additional foam-material element (11′), which is held on the supporting part (11) by means of a lug (11″), serves for redirecting the air, and consequently for reducing noise.

FIG. 5 illustrates a typical air flow through the blower box (5). According to A, recirculated air is sucked in through an intake opening in the rear part (16) and, according to B, flows through the opening (11.1) in the supporting part (11). According to C, the air flow is redirected vertically through a chamber (17.1) in the front part (17) and passes through the opening (11.2) in the supporting part (11) back into the rear part (16), into the air chamber in the region (16.1). There, the air is redirected diagonally and, according to D, flows through the opening (11.3) and, diagonally redirected further according to E, in the region (9′) flows into the blower (9). The air transported by the blower (9) leaves the blower through the air outlet (9″) according to F in the region (17.2) in the front part (17) and further according to G through the opening (11.4) in the supporting part (11). According to H, the air flow reaches the rear part (16) in the region (16.2), flows there through a flow measuring device and is redirected again according to I, and leaves the rear part (16) to a sound damper or to the respiratory gas humidifier.

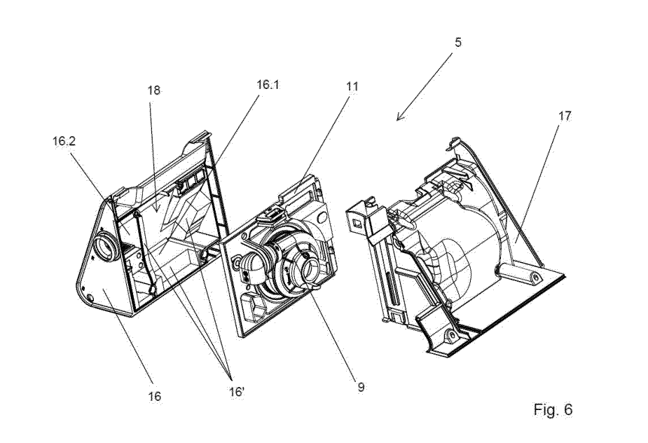

FIG. 6 shows the components according to FIG. 5 for a more detailed explanation of the mechanical construction without the flow arrows of the gas flow being depicted.

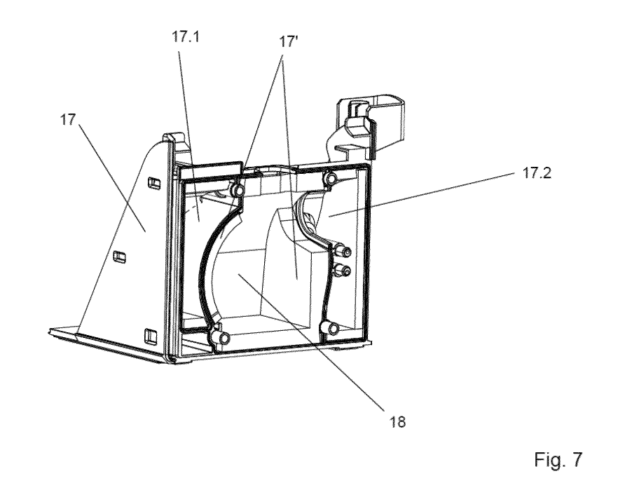

FIG. 7 shows the interior housing space (18) in the front part (17), with inserted sound-damping materials (17′) and the air chambers (17.1) and (17.2).

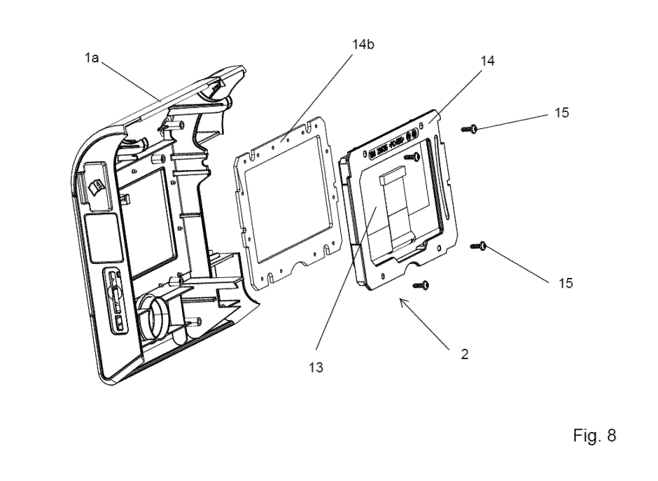

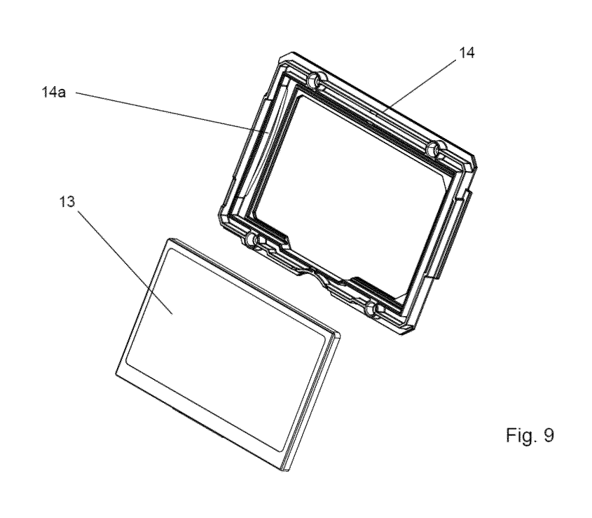

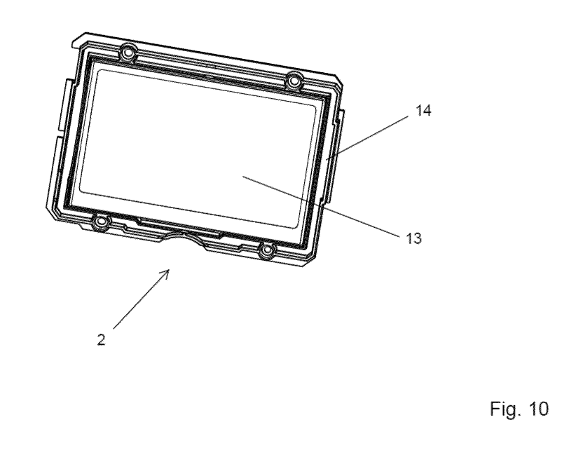

According to the embodiment in FIG. 8, the indicating device (2) is formed as a display (13). The display (13) is designed in the manner of a panel and is fixed within the housing (5) by means of a frame-like securing element (14). It is preferably envisaged to insert the display (13) into the securing element (14) and fix it in the securing element (14) by means of a clasp (14a). This serves for better fixing of the display in the housing, since the display is fitted into the housing while inverted, and without a clasp may slip. The securing element (14) is also designed in such a way that it can compensate for production tolerances of the display (13) by varying tensioning of the clasp (14a). The individual parts are represented in FIG. 9 and the preassembled unit comprising the display (13) and the securing element (14) is represented in FIG. 10. The preassembled unit is braced against the housing (5) by using screws (15). A seal (14b) is preferably arranged between the display (13) and the housing (1a). If a different display (13) is used, for example from a different manufacturer, in this embodiment only the securing element (14) need be adapted to the display, the housing structure can be retained.

FIG. 11 shows an embodiment in which the respirator (1) is provided with an additional element (8) formed as a communication module. The additional element (8) serves here for the connection to a polysomnography device (PSD) (19). Coupling of the PSD (19) may take place by using a cable (20), which can be coupled to an interface (22) of the additional element (8) by means of a connector (21).

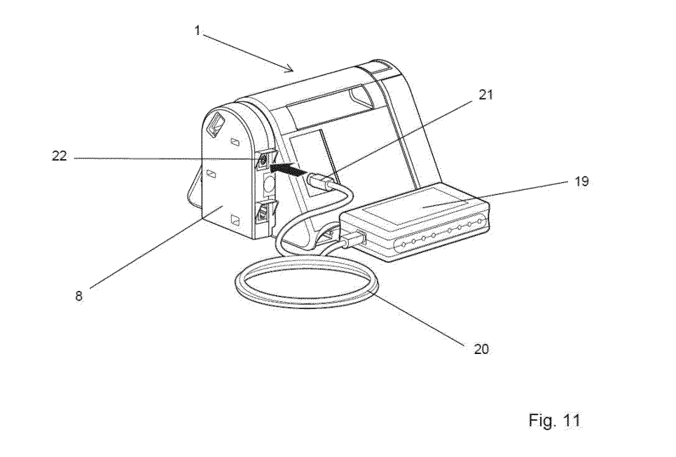

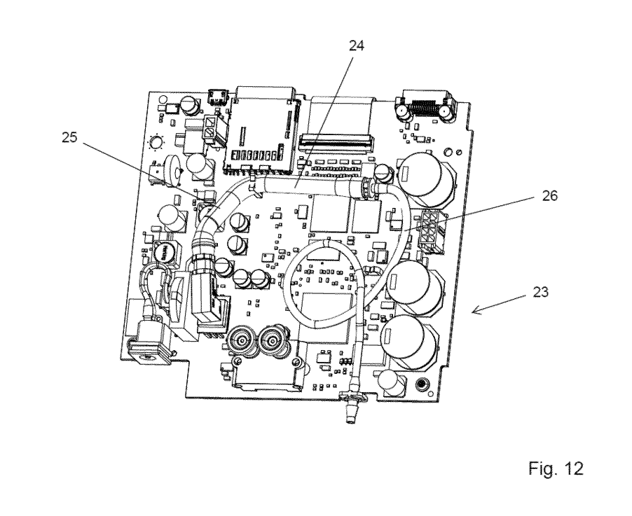

FIG. 12 shows a plan view of a control board (23), which can be arranged within the front unit (1a) of the respirator (1). The control board (23) assists a pressure measurement and is provided with a connectable tube (24) for passing on the pressure.

According to the exemplary embodiment represented, the tube (24) consists of a first tube portion (25) of a first diameter and a second tube portion (26) of a second diameter. The first diameter is greater than the second diameter.

The combination of the first tube portion (25) and the second tube portion (26) provides signal filtering, which in particular filters out harmonics present in the pressure signal. As an alternative to the filter taking a mechanical form provided by the tube portions (25, 26), electrical, electronic or software-based filtering is also possible in principle.

In combination with at least one of the components and/or method steps explained above, it is in particular envisaged to form the communication interface (6) as a USB port or micro-USB port. In addition, it is in particular also envisaged to provide the communication interface (6) with a detector or connect it to a detector that detects whether a cable and/or a connector is connected to the communication interface (6).

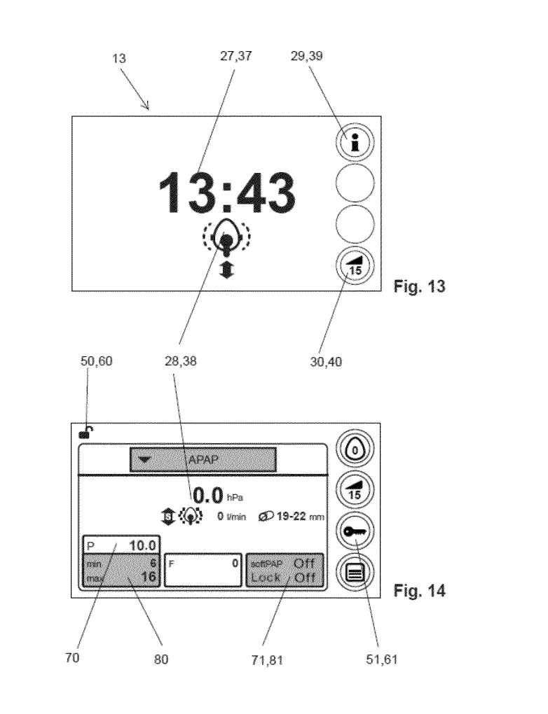

FIG. 13 illustrates a possible indication of the display (13), in a basic state of the respirator (1). Since the respirator is often placed on a bedside table of the patient overnight, in the exemplary embodiment represented the display (13) indicates the current time of day (27, 37). In addition, an indication of a mask leak-tightness test (28, 38) carried out during the therapy takes place by a symbolic visualization in the patient menu. The corresponding functional symbol can be seen underneath the indicated time of day.

The display (13) is formed here as a user interface, for example as a touchscreen, of the respirator (1). A number of input buttons (27, 28, 29, 30) configured for receiving a user input are provided in the region of the display (13). The display (13) is connected by logic circuitry to the buttons (27-30) and has a number of information fields (37, 38, 39, 40), wherein each information field is assigned to a corresponding input button (27, 28, 29, 30) and is configured for indicating graphic items of information that specify various functions of the corresponding input buttons (27, 28, 29, 30). The input buttons (27, 28, 29, 30) are touch-sensitive areas in the region of the display (13).

According to the invention, only one further operating element (3) is provided (see FIGS. 1, 2 and 3). This operating element (3) can be operated mechanically, and consequently differs from the touch areas (27, 28, 29, 30) on the display. According to the invention, the respirator is switched on and off by means of the operating element (3), so that a basic patient-specific therapy can be activated by means of just one button. For this purpose, the operating element (3) is connected by logic circuitry to the blower motor and activates it, and also the memory, in order to retrieve and apply stored therapy data, such as pressure values. In addition, adaptation of individual values, such as pressure values, may also take place by means of the input buttons (27, 28, 29, 30). However, according to the invention, it is not necessary to use the input buttons (27, 28, 29, 30) to operate the touchscreen (13) in order to start the therapy.

FIG. 14 shows an indication of the display (13) in an active basic state of the respirator after the input screen according to FIG. 13 has been left. The representation shows, after actuating the input button (28), in particular the carrying out of a test procedure for the leak-tightness of the mask during the therapy in the expert menu, with an indication of 1/min, and in a state for adjusting the pressure (80), in order also to test the leak-tightness of the mask at higher pressure values. This corresponds to the tile-like representation of symbols in the display indication at the bottom left with P min/max. In the region of the display (13), a number of input buttons (27 . . . 50, 51) configured for receiving a user input are again provided. The display (13) is connected by logic circuitry to the buttons (27-51) and has a number of assigned information fields (37 . . . 61), wherein each information field is assigned to a corresponding input button and configured for indicating graphic items of information that specify various functions of the corresponding input buttons. The input buttons are touch-sensitive areas in the region of the display (13). According to the invention, at least one information field (60, 61) with an assigned input button (50, 51) is provided.

The information field (60, 61) is designed to indicate graphically whether or not a selected value (80, 81) concerning a parameter or a setting of the respirator can be changed by means of at least one input button (70, 71). The information field (60) illustrates here that it is possible for the values to be changed (open lock). Changing of this state can be achieved by means of confirmation of the input button (51), which is assigned to the information field (61). After corresponding actuation of the input button (51), the information field (60) visualizes a closed lock and at least selected values (80, 81) that concern the operation of the respirator can no longer be changed.

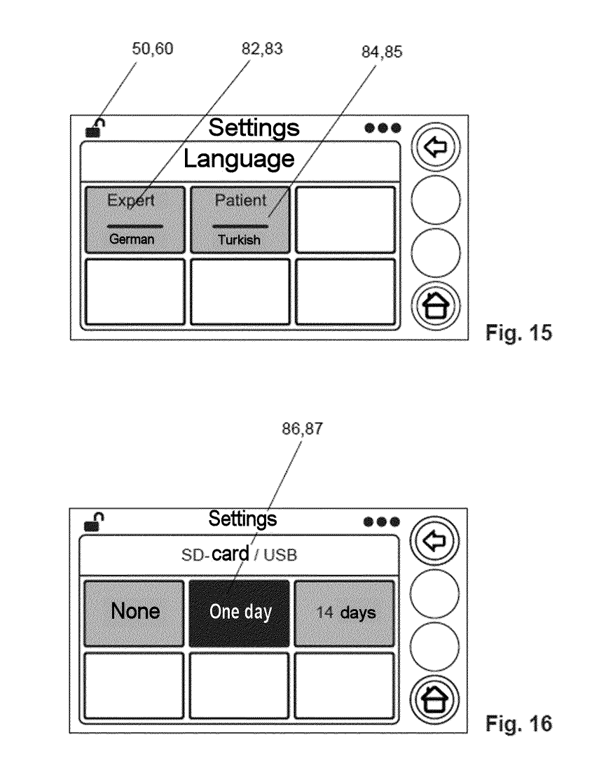

FIG. 15 shows an indication of the display (13) to assist an input for the selection of a language used in the patient menu and/or expert menu. In the region of the display (13), a number of input buttons (27 . . . 50 . . . 82, 84) configured for receiving a user input are again provided. The display (13) is connected by logic circuitry to the buttons (27-84) and has a number of assigned information fields (37 . . . 85), wherein each information field is assigned to a corresponding input button and configured for indicating graphic items of information that specify various functions of the corresponding input buttons. The input buttons are touch-sensitive areas in the region of the display (13). According to the invention, at least one information field with an assigned input button is provided. The information field (60) is designed to indicate graphically whether or not a selected value (82, 84) concerning a parameter or a setting of the respirator can be changed by means of at least one input button (83, 85). The information field (60) illustrates here that it is possible for the values to be changed (open lock); which symbolizes that the device is in the expert mode. The expert mode is characterized in that many basic settings can be performed here, whereas in the patient mode only comfort functions for example can be changed. In the expert menu, the language can be set.

The possibility of selecting different languages for the patient menu and the expert menu makes it possible for the device to be operated safely by the expert and the patient. The different language instructions in the menus are independent of one another. The invention makes allowance for the situation in which the doctor and the patient speak different languages. In the exemplary embodiment represented, German has been chosen for the expert menu and Turkish as the language for the patient menu. For this purpose, the language for the patient menu is selected and presented by means of the input button (84). The input button is connected by logic circuitry to a language selection menu, in which the user can select the language. The input button (84) is also connected by logic circuitry to the language memory. After confirmation of the chosen language, it is loaded from the memory and used. The language for the expert mode is selected and set in the same way by means of the button (82). Such a menu structure helps to ensure that the respirator (1) can be safely operated even by persons who for example only have a partial command of the German language. A specialist expert can also access the parameter settings that can be changed in his own selected language.

According to the representation of the display (13) in FIG. 16, the operating structure offers the possibility of storing the therapy data on a memory card. This may be for example an SD card or a USB memory. The corresponding storage capability ensures that no therapy data can be lost. The data concerned are additionally always stored on the device. A copy of the data stored in the device is written to the memory card by the menu when it is correspondingly activated. The extent of the data to be transferred to the memory card can be set. In the exemplary embodiment represented, for example, the data for one day or for 14 days can be stored. In the region of the display (13), a number of input buttons (27 . . . 86) configured for receiving a user input are again provided. The display (13) is connected by logic circuitry to the buttons (27-86) and has a number of assigned information fields (37 . . . 87), wherein each information field is assigned to a corresponding input button and configured for indicating graphic items of information that specify various functions of the corresponding input buttons. The input buttons are touch-sensitive areas in the region of the display (13). According to the invention, at least one information field with an assigned input button is provided. The information field (87) is designed to indicate graphically how many days with memory data are to be transferred to the external memory (an SD card or a USB memory). The input button (86) is connected by logic circuitry to the memory for therapy data. Selecting the button (86) causes the data of a day (the last day) to be copied from the memory for therapy data to the external memory.

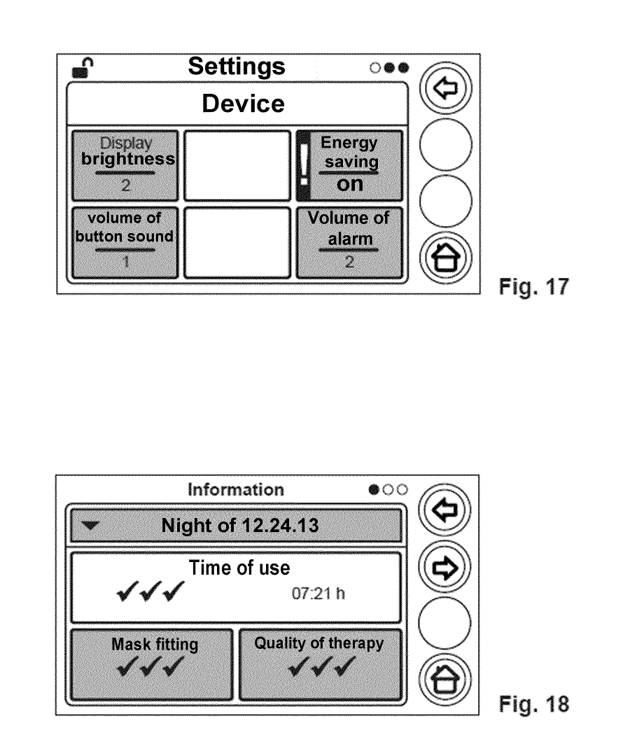

According to the indication of the display (13) that is represented in FIG. 17, it is possible that, after a predeterminable time, the respirator (1) is switched to an energy-saving mode. A corresponding extent of functions within the energy-saving mode can be selected by means of the represented visualization on the display (13) in the form of a touchscreen. The brightness of the display can also be set in stages. The volume for the sound of an alarm can also be set in stages.

FIG. 18 visualizes an indication of the display (13) for transmitting to a patient information concerning the quality of the therapy carried out. For this purpose, the respirator statistically prepares the data for a selected time period (here one night) and indicates the time of use. The time of use is also assessed qualitatively and a symbolic graphic output that respresents or illustrates a qualitative statement about the time of use is generated, and indicated alongside the time of use. Rules for this are stored in the device. For example, a time of use of less than 3 hours per night is assessed to be insufficient, in which case a symbolic graphic output that represents or illustrates a qualitative statement finding the time of use to be “insufficient” is generated and indicated alongside the time of use. Similarly, the quality of the therapy is qualitatively assessed and a symbolic graphic output that represents or illustrates a qualitative statement about the quality of the therapy is generated and indicated. Rules for this are stored in the device. For example, an AHI of less than 1 per hour is assessed as sufficient, in which case a symbolic graphic output that represents or illustrates a qualitative statement finding the time of use to be “sufficient” is generated and indicated. Preferably, the assessment of the quality of the therapy is based on multiple measurement data.

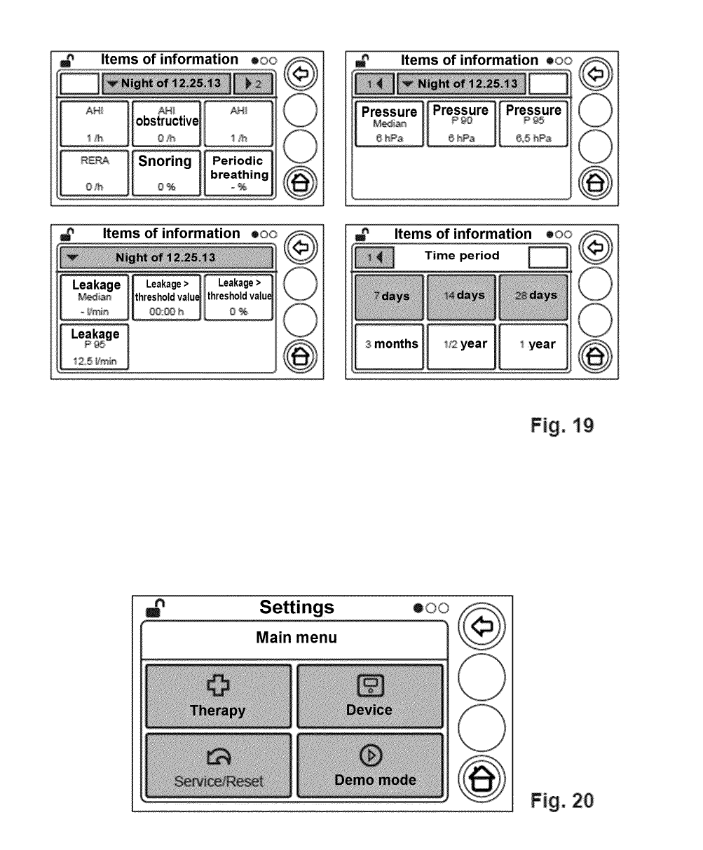

FIG. 19 illustrates various indications of the display (13), to transmit to a patient further qualitative items of information concerning the therapy carried out. For example, according to FIG. 18, the assessment of the quality of the therapy is based on multiple measurement data, such as snoring and/or a periodic breathing component and/or average therapy pressure and/or leakage. The determination and storage of the corresponding data takes place online in the device. For example, it is possible to visualize the quality of the therapy for certain time periods of up to one year. An assessment of the therapy is thus also possible without special therapy software.

According to the indication of the display (13) in FIG. 20, it is possible to activate demonstration visualizations by means of the demo mode array of buttons, with which the functionalities are explained to a user. For example, it is possible to activate a demonstration for the carrying out of measuring procedures. It is likewise possible to select different menu levels or representations of functions for demonstration purposes.

While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

To sum up, the present invention provides:
1. A device for artificial respiration, which has a blower connected to a control, wherein both the control and the blower are arranged in a housing, and wherein the control is connected to at least one indicating device and also at least one operating element, characterized in that the indicating device (2) is formed as a display (13) that has a touchscreen function and, using a symbol-based operating structure, can be used as an input device (2) in such a way that respective symbols visualize an assigned function and that a hierarchically structured operating capability is generated by the control.
2. The device according to item 1, wherein different languages can be selected by means of the display (13).
3. The device according to item 1 and/or 2, wherein a possibility for the selection of different languages is provided for a patient menu and an expert menu, wherein the language for the menu concerned is selected by means of the input button (82, 84) and the input button is connected by logic circuitry to a language selection menu, in which the user can select the language, wherein the input button (82, 84) is also connected by logic circuitry to the language memory and wherein, after confirmation of the language selected, this language is loaded from the memory and used.
4. The device according to item 2 and/or 3, wherein a number of input buttons (27 . . . 50 . . . 82, 84) configured for receiving a user input are provided in the region of the display (13), wherein the display (13) is connected by logic circuitry to the buttons (27-84) and has a number of assigned information fields (37 . . . 85), wherein each information field is assigned to a corresponding input button and configured for indicating graphic items of information that specify various functions of the corresponding input buttons, wherein the information field (60) is designed to indicate graphically whether or not a selected value (82, 84) concerning a parameter or a setting of the respirator can be changed by means of at least one input button (83, 85).
5. The device according to at least one of the preceding items, wherein the display (13) is connected to an evaluation unit for providing at least one item of qualitative therapy information.
6. The device according to at least one of the preceding items, wherein the respirator (1) has an internal data memory for permanently storing operating data.
7. The device according to at least one of the preceding items, wherein selectable operating data can be stored on a memory card.
8. The device according to at least one of the preceding items, wherein a number of input buttons (27 . . . 86) configured for receiving a user input are provided in the region of the display (13), wherein the display (13) is connected by logic circuitry to the buttons (27-86) and has a number of assigned information fields (37 . . . 87), wherein each information field is assigned to a corresponding input button and configured for indicating graphic items of information that specify various functions of the corresponding input buttons, wherein the information field (87) is designed to indicate graphically how many days with memory data are to be transferred to the external memory (an SD card or USB memory).
9. The device according to at least one of the preceding items, wherein the respirator (1) is designed for carrying out a leak-tightness test of a respiration mask and in that a corresponding test result can be visualized in the region of the display (13).
10. The device according to item 9, wherein a pressure used while carrying out the test for the leak-tightness of the mask can be preset by means of the display (13).
11. The device according to item 9 and/or 10, wherein, after actuating the input button (28), in particular the carrying out of a test procedure for the leak-tightness of the mask takes place during the therapy in the expert menu, wherein a number of input buttons (27 . . . 50, 51) configured for receiving a user input are provided in the region of the display (13) and wherein the display (13) is connected by logic circuitry to the buttons (27-51) and has a number of assigned information fields (37 . . . 61), wherein each information field is assigned to a corresponding input button and configured for indicating graphic items of information that specify various functions of the corresponding input buttons, wherein at least one information field (60, 61) is provided with an assigned input button (50, 51), wherein the information field (60, 61) is designed to indicate graphically whether or not a selected value (80, 81) concerning a parameter or a setting of the respirator can be changed by means of at least one input button (70, 71) and a change of this state can be achieved by means of actuating the input button (51) that is assigned to the information field (61), wherein, after corresponding actuation of the input button (51), the information field (60) visualizes a closed state and, after that, at least selected values (80, 81) that concern the operation of the respirator can no longer be changed.
12. The device according to at least one of the preceding items, wherein at least one energy-saving option can be selected and can be configured by means of the display (13).
13. The device according to at least one of the preceding items, wherein at least one item of information with respect to the function of the device and/or operating the device can be visualized by means of the display (13).
14. A method for indicating user interface information for a respirator, which has a blower connected to a control, wherein both the control and the blower are arranged in a housing, and wherein the control is connected to at least one indicating device and also at least one operating element, wherein the indicating device (2) is formed as a display (13) that has a touchscreen function and, using a symbol-based operating structure, can be used as an input device (2) in such a way that respective symbols visualize an assigned function and that a hierarchically structured operating capability is generated by the control.
15. The method according to item 14, whreein a number of input buttons (27 . . . 50, 51) configured for receiving a user input are provided in the region of the display (13) and wherein the display (13) is connected by logic circuitry to the buttons (27-51) and has a number of assigned information fields (37 . . . 61), wherein each information field is assigned to a corresponding input button and configured for indicating graphic items of information that specify various functions of the corresponding input buttons, wherein at least one information field (60, 61) with an assigned input button (50, 51) is provided, wherein the information field (60, 61) is designed to indicate graphically whether a selected value (80, 81) concerning a parameter or a setting of the respirator can be changed by means of at least one input button (70, 71).

What is claimed is:

1. A device for artificial respiration, wherein the device comprises a blower connected to a control, both the control and the blower being arranged in a housing and the control being connected to at least one indicating device and also at least one operating element, and wherein the indicating device is formed as a display that has a touchscreen function and, using a symbol-based operating structure, can be used as an input device in such a way that respective symbols visualize an assigned function and that a hierarchically structured operating capability is generated by the control.

2. The device of claim 1, wherein different languages can be selected by means of the display.

3. The device of claim 1, wherein a possibility for selecting different languages is provided for a patient menu and an expert menu, wherein a language for the menu concerned is selected by an input button and the input button is connected by logic circuitry to a language selection menu, in which the user can select the language, wherein the input button is also connected by logic circuitry to a language memory and wherein, after confirmation of a language selected, this language is loaded from the memory and used.

4. The device of claim 2, wherein a number of input buttons configured for receiving a user input are provided in a region of the display, wherein the display is connected by logic circuitry to the buttons and has a number of assigned information fields, wherein each information field is assigned to a corresponding input button and configured for indicating graphic items of information that specify various functions of corresponding input buttons, and wherein an information field is designed to indicate graphically whether or not a selected value concerning a parameter or a setting of the respirator can be changed by means of at least one input button.

5. The device of claim 1, wherein the display is connected to an evaluation unit for providing at least one item of qualitative therapy information.

6. The device of claim 1, wherein the respirator comprises an internal data memory for permanently storing operating data.

7. The device of claim 1, wherein selectable operating data can be stored on a memory card.

8. The device of claim 1, wherein a number of input buttons configured for receiving a user input are provided in a region of the display, wherein the display is connected by logic circuitry to the input buttons and comprises a number of assigned information fields, wherein each information field is assigned to a corresponding input button and configured for indicating graphic items of information that specify various functions of the corresponding input buttons, wherein the information field is designed to indicate graphically how many days with memory data are to be transferred to an external memory.

9. The device of claim 8, wherein the external memory is an SD card or USB memory.

10. The device of claim 1, wherein the respirator is designed for carrying out a leak-tightness test of a respiration mask and a corresponding test result can be visualized in a region of the display.

11. The device of claim 10, wherein a pressure used while carrying out the test for the leak-tightness of the mask can be preset by means of the display.

12. The device of claim 10, wherein, after actuating an input button, carrying out of a test procedure for leak-tightness of the mask takes place during therapy in the expert menu, wherein a number of input buttons configured for receiving a user input are provided in a region of the display and wherein the display is connected by logic circuitry to the input buttons and has a number of assigned information fields, wherein each information field is assigned to a corresponding input button and configured for indicating graphic items of information that specify various functions of corresponding input buttons, wherein at least one information field is provided with an assigned input button, wherein the information field is designed to indicate graphically whether or not a selected value concerning a parameter or a setting of the respirator can be changed by means of at least one input button and a change of this state can be achieved by actuating an input button that is assigned to the information field, wherein, after corresponding actuation of the input button, the information field visualizes a closed state and, after that, at least selected values that concern an operation of the respirator can no longer be changed.

13. The device of claim 1, wherein at least one energy-saving option can be selected and can be configured by means of the display.

14. The device of claim 1, wherein at least one item of information with respect to a function of the device and/or operating the device can be visualized by means of the display.

15. A method for indicating user interface information for a respirator comprising a blower connected to a control, both the control and the blower being arranged in a housing, and the control being connected to at least one indicating device and also at least one operating element, wherein the indicating device is formed as a display that has a touchscreen function and, using a symbol-based operating structure, can be used as an input device in such a way that respective symbols visualize an assigned function and that a hierarchically structured operating capability is generated by the control.

16. The method of claim 15, wherein a number of input buttons configured for receiving a user input are provided in a region of the display and wherein the display is connected by logic circuitry to the input buttons and has a number of assigned information fields, wherein each information field is assigned to a corresponding input button and configured for indicating graphic items of information that specify various functions of corresponding input buttons, wherein at least one information field with an assigned input button is provided, wherein the information field is designed to indicate graphically whether a selected value concerning a parameter or a setting of the respirator can be changed by means of at least one input button.
